(19) United States
(12) Patent Application Publication
(10) Pub. No.: US 2021/0114912 A1
(43) Pub. Date: Apr. 22, 2021

(54) METHODS FOR ANALYZING A GLASS MELT COMPOSITION

(71) Applicant: Corning Incorporated, Corning, NY (US)

(72) Inventors: Miki Eugene Kunitake, Elmira, NY (US); Irene Mona Peterson, Elmira Heights, NY (US); Michael Clement Ruotolo, Jr., Corning, NY (US); Elizabeth Mary Sturdevant, Corning, NY (US); Matthew Scott Weibel, Corning, NY (US)

(21) Appl. No.: 16/494,048

(22) PCT Filed: Mar. 13, 2018
(86) PCT No.: PCT/US2018/022154

§ 371 Date: Sep. 13, 2019

Related U.S. Application Data
(60) Provisional application No. 62/471,475, filed on Mar. 15, 2017.

(51) Int. Cl. C03B 5/24; G01N 21/3504; G01N 30/72; G01N 33/00

(52) U.S. Cl. CPC C03B 5/24; G01N 21/3504; G01N 2021/3595; G01N 33/0036; G01N 30/72

(57) ABSTRACT

A method for analyzing a glass melt composition, in the manufacture of glass articles, includes sampling a gas composition comprising at least one gaseous species generated from the glass melt composition during a melting operation by taking a plurality of measurements of the gas composition over a period of time. The method also includes analyzing the sampled composition to determine an amount or concentration of the at least one gaseous species in the gas composition as a function of time.

This application claims the benefit of priority under 35 U.S.C. § 119 of U.S. Provisional Application Ser. No. 62/471,475, filed on Mar. 15, 2017, the content of which is relied upon and incorporated herein by reference in its entirety.

### FIELD

The present disclosure relates generally to methods for analyzing a glass melt composition and more particularly to methods for analyzing a glass melt composition by analyzing gaseous species generated from the glass melt composition.

### BACKGROUND

In the production of glass articles, such as glass sheets for display applications, including televisions and hand held devices, such as telephones and tablets, raw materials are typically melted into molten glass, which is in, turn, formed and cooled to make the intended glass article. During the process of melting the raw materials, at least some material initially present in the raw materials may be converted to a gaseous phase during a melting operation, such as through decomposition reactions occurring in a glass melt composition. Such gaseous species may, in turn, comprise a portion of exhaust gasses exiting a component of a glass manufacturing apparatus, such as a glass melting vessel.

In addition, there are many variables that can affect the processing of the glass melt composition as it is converted from raw materials to molten glass. Such variables can, in turn, affect properties of the glass articles ultimately produced. Monitoring these variables in real time, which, for example, include batch material segregation prior to the melting process, batch to melt conversion time for specific batch materials, and batch material decomposition reactions during the melting process, tends to be very difficult. Developing a greater understanding of these variables can provide valuable insights for fine tuning and controlling various processes associated with the production of glass articles, which can, in turn, lead to the manufacture of higher quality glass articles and/or glass articles that are produced more efficiently.

### SUMMARY

Embodiments disclosed herein include a method for analyzing a glass melt composition. The method includes sampling a gas composition comprising at least one gaseous species generated from the glass melt composition during a melting operation by taking a plurality of measurements of the gas composition over a period of time. The method also includes analyzing the sampled composition to determine an amount or concentration of the at least one gaseous species in the gas composition as a function of time.

Embodiments disclosed herein also include a method of making a glass article. The method includes sampling a gas composition comprising at least one gaseous species generated from a glass melt composition during a melting operation by taking a plurality of measurements of the gas composition over a period of time. The method also includes analyzing the sampled composition to determine an amount or concentration of the at least one gaseous species in the gas composition as a function of time. In addition, the method includes forming the glass article from the glass melt composition.

Additional features and advantages of the embodiments disclosed herein will be set forth in the detailed description which follows, and in part will be readily apparent to those skilled in the art from that description or recognized by practicing the disclosed embodiments as described herein, including the detailed description which follows, the claims, as well as the appended drawings.

It is to be understood that both the foregoing general description and the following detailed description present embodiments intended to provide an overview or framework for understanding the nature and character of the claimed embodiments. The accompanying drawings are included to provide further understanding, and are incorporated into and constitute a part of this specification. The drawings illustrate various embodiments of the disclosure, and together with the description serve to explain the principles and operations thereof.

### BRIEF DESCRIPTION OF THE DRAWINGS

FIG. 1 is a schematic view of an example fusion down draw glass making apparatus and process;

FIG. 2 is a schematic view of an example glass melting and gas sampling and analyzing system;

FIG. 3 is a schematic view of an example pollution abatement system that includes gas sampling and analysis;

FIG. 4 is a chart showing HCl concentration as a function of time at three different sampling locations in a pollution abatement system;

FIG. 5 is a chart showing NO concentration as a function of time following introduction of nitrate-containing and nitrate-free batch into a glass melting vessel;

FIG. 6 is a chart showing SO₂ concentration as a function of time following introduction of boron-containing raw materials at different ratios into a glass melting vessel; and

FIG. 7 is a chart showing NO concentration as a function of time following introduction of nitrate-containing and nitrate-free batch into a glass melting vessel.

### DETAILED DESCRIPTION

Reference will now be made in detail to the present preferred embodiments of the present disclosure, examples of which are illustrated in the accompanying drawings. Whenever possible, the same reference numerals will be used throughout the drawings to refer to the same or like parts. However, this disclosure may be embodied in many different forms and should not be construed as limited to the embodiments set forth herein.

Ranges can be expressed herein as from “about” one particular value, and/or to “about” another particular value. When such a range is expressed, another embodiment includes from the one particular value and/or to the other particular value. Similarly, when values are expressed as approximations, for example by use of the antecedent “about,” it will be understood that the particular value forms another embodiment. It will be further understood that the endpoints of each of the ranges are significant both in relation to the other endpoint, and independently of the other endpoint.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

As used herein, the term “glass melt composition” refers to a composition from which a glass article is made, wherein the composition may exist in any state between and including a substantially solid state and a substantially liquid state, such any state between and including raw materials and molten glass, including any degree of partial melting there between.

As used herein, the term “melting operation” refers to an operation in which a glass melt composition is heated from a substantially solid state to a substantially liquid state so as to convert raw materials into molten glass.

As used herein, the term “gaseous species generated from the glass melt composition” refers to material initially present in raw materials of the glass melt composition that is converted to a gaseous phase during a melting operation.

As used herein, the term “processing condition of the glass melt composition” refers to any condition under which a glass melt composition is processed, including any condition under which the glass melt composition exists between and including a substantially solid state and a substantially liquid state, including any condition before, during, or after a melting operation.

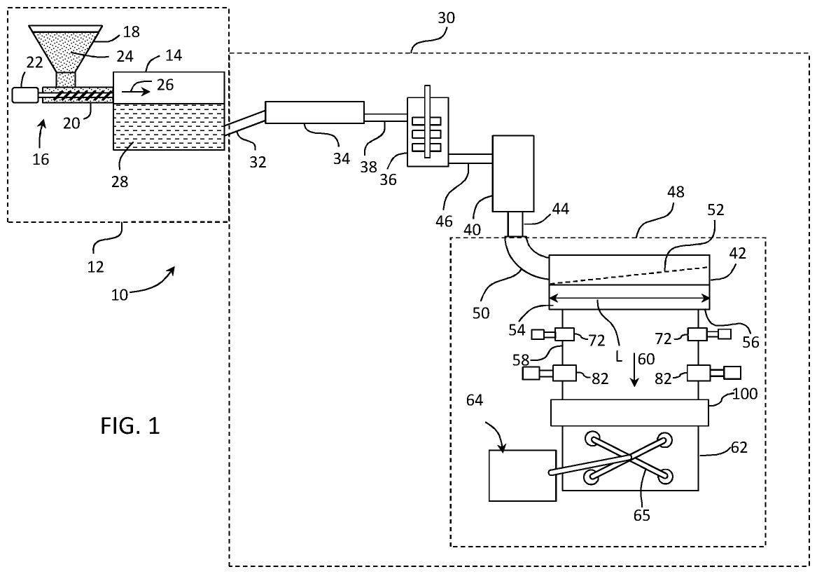

Shown in FIG. 1 is an exemplary glass manufacturing apparatus 10. In some examples, the glass manufacturing apparatus 10 can comprise a glass melting furnace 12 that can include a melting vessel 14. In addition to melting vessel 14, glass melting furnace 12 can optionally include one or more additional components such as heating elements (e.g., combustion burners or electrodes) that heat raw materials and convert the raw materials into molten glass. In further examples, glass melting furnace 12 may include thermal management devices (e.g., insulation components) that reduce heat lost from a vicinity of the melting vessel. In still further examples, glass melting furnace 12 may include electronic devices and/or electromechanical devices that facilitate melting of the raw materials into a glass melt. Still further, glass melting furnace 12 may include support structures (e.g., support chassis, support member, etc.) or other components.

Glass melting vessel 14 is typically comprised of refractory material, such as a refractory ceramic material, for example a refractory ceramic material comprising alumina or zirconia. In some examples glass melting vessel 14 may be constructed from refractory ceramic bricks. Specific embodiments of glass melting vessel 14 will be described in more detail below.

In some examples, the glass melting furnace may be incorporated as a component of a glass manufacturing apparatus to fabricate a glass substrate, for example a glass ribbon of a continuous length. In some examples, the glass melting furnace of the disclosure may be incorporated as a component of a glass manufacturing apparatus comprising a slot draw apparatus, a float bath apparatus, a down-draw apparatus such as a fusion process, an up-draw apparatus, a press-rolling apparatus, a tube drawing apparatus or any other glass manufacturing apparatus that would benefit from the aspects disclosed herein. By way of example, FIG. 1 schematically illustrates glass melting furnace 12 as a component of a fusion down-draw glass manufacturing apparatus 10 for fusion drawing a glass ribbon for subsequent processing into individual glass sheets.

The glass manufacturing apparatus 10 (e.g., fusion down-draw apparatus 10) can optionally include an upstream glass manufacturing apparatus 16 that is positioned upstream relative to glass melting vessel 14. In some examples, a portion of, or the entire upstream glass manufacturing apparatus 16, may be incorporated as part of the glass melting furnace 12.

As shown in the illustrated example, the upstream glass manufacturing apparatus 16 can include a storage bin 18, a raw material delivery device 20 and a motor 22 connected to the raw material delivery device. Storage bin 18 may be configured to store a quantity of raw materials 24 that can be fed into melting vessel 14 of glass melting furnace 12, as indicated by arrow 26. Raw materials 24 typically comprise one or more glass forming metal oxides and one or more modifying agents. In some examples, raw material delivery device 20 can be powered by motor 22 such that raw material delivery device 20 delivers a predetermined amount of raw materials 24 from the storage bin 18 to melting vessel 14. In further examples, motor 22 can power raw material delivery device 20 to introduce raw materials 24 at a controlled rate based on a level of molten glass sensed downstream from melting vessel 14. Raw materials 24 within melting vessel 14 can thereafter be heated to form molten glass 28.

Glass manufacturing apparatus 10 can also optionally include a downstream glass manufacturing apparatus 30 positioned downstream relative to glass melting furnace 12. In some examples, a portion of downstream glass manufacturing apparatus 30 may be incorporated as part of glass melting furnace 12. In some instances, first connecting conduit 32 discussed below, or other portions of the downstream glass manufacturing apparatus 30, may be incorporated as part of glass melting furnace 12. Elements of the downstream glass manufacturing apparatus, including first connecting conduit 32, may be formed from a precious metal. Suitable precious metals include platinum group metals selected from the group of metals consisting of platinum, iridium, rhodium, osmium, ruthenium and palladium, or alloys thereof. For example, downstream components of the glass manufacturing apparatus may be formed from a platinum-rhodium alloy including from about 70 to about 90% by weight platinum and about 10% to about 30% by weight rhodium. However, other suitable metals can include molybdenum, palladium, rhenium, tantalum, titanium, tungsten and alloys thereof.

Downstream glass manufacturing apparatus 30 can include a first conditioning (i.e., processing) vessel, such as fining vessel 34, located downstream from melting vessel 14 and coupled to melting vessel 14 by way of the above-referenced first connecting conduit 32. In some examples, molten glass 28 may be gravity fed from melting vessel 14 to fining vessel 34 by way of first connecting conduit 32. For instance, gravity may cause molten glass 28 to pass through an interior pathway of first connecting conduit 32 from melting vessel 14 to fining vessel 34. It should be understood, however, that other conditioning vessels may be positioned downstream of melting vessel 14, for example between melting vessel 14 and fining vessel 34. In some embodiments, a conditioning vessel may be employed between the melting vessel and the fining vessel wherein molten glass from a primary melting vessel is further heated to continue the melting process, or cooled to a temperature lower than the temperature of the molten glass in the melting vessel before entering the fining vessel.

Bubbles may be removed from molten glass 28 within fining vessel 34 by various techniques. For example, raw materials 24 may include multivalent compounds (i.e. fining agents) such as tin oxide that, when heated, undergo a chemical reduction reaction and release oxygen. Other suitable fining agents include without limitation arsenic, antimony, iron and cerium. Fining vessel 34 is heated to a temperature greater than the melting vessel temperature, thereby heating the molten glass and the fining agent. Oxygen bubbles produced by the temperature-induced chemical reduction of the fining agent(s) rise through the molten glass within the fining vessel, wherein gases in the molten glass produced in the melting furnace can diffuse or coalesce into the oxygen bubbles produced by the fining agent. The enlarged gas bubbles can then rise to a free surface of the molten glass in the fining vessel and thereafter be vented out of the fining vessel. The oxygen bubbles can further induce mechanical mixing of the molten glass in the fining vessel.

Downstream glass manufacturing apparatus 30 can further include another conditioning vessel such as a mixing vessel 36 for mixing the molten glass. Mixing vessel 36 may be located downstream from the fining vessel 34. Mixing vessel 36 can be used to provide a homogenous glass melt composition, thereby reducing cords of chemical or thermal inhomogeneity that may otherwise exist within the fined molten glass exiting the fining vessel. As shown, fining vessel 34 may be coupled to mixing vessel 36 by way of a second connecting conduit 38. In some examples, molten glass 28 may be gravity fed from the fining vessel 34 to mixing vessel 36 by way of second connecting conduit 38. For instance, gravity may cause molten glass 28 to pass through an interior pathway of second connecting conduit 38 from fining vessel 34 to mixing vessel 36. It should be noted that while mixing vessel 36 is shown downstream of fining vessel 34, mixing vessel 36 may be positioned upstream from fining vessel 34. In some embodiments, downstream glass manufacturing apparatus 30 may include multiple mixing vessels, for example a mixing vessel upstream from fining vessel 34 and a mixing vessel downstream from fining vessel 34. These multiple mixing vessels may be of the same design, or they may be of different designs.

Downstream glass manufacturing apparatus 30 can further include another conditioning vessel such as delivery vessel 40 that may be located downstream from mixing vessel 36. Delivery vessel 40 may condition molten glass 28 to be fed into a downstream forming device. For instance, delivery vessel 40 can act as an accumulator and/or flow controller to adjust and/or provide a consistent flow of molten glass 28 to forming body 42 by way of exit conduit 44. As shown, mixing vessel 36 may be coupled to delivery vessel 40 by way of third connecting conduit 46. In some examples, molten glass 28 may be gravity fed from mixing vessel 36 to delivery vessel 40 by way of third connecting conduit 46. For instance, gravity may drive molten glass 28 through an interior pathway of third connecting conduit 46 from mixing vessel 36 to delivery vessel 40.

Downstream glass manufacturing apparatus 30 can further include forming apparatus 48 comprising the above-referenced forming body 42 and inlet conduit 50. Exit conduit 44 can be positioned to deliver molten glass 28 from delivery vessel 40 to inlet conduit 50 of forming apparatus 48. For example in examples, exit conduit 44 may be nested within and spaced apart from an inner surface of inlet conduit 50, thereby providing a free surface of molten glass positioned between the outer surface of exit conduit 44 and the inner surface of inlet conduit 50. Forming body 42 in a fusion down draw glass making apparatus can comprise a trough 52 positioned in an upper surface of the forming body and converging forming surfaces 54 that converge in a draw direction along a bottom edge 56 of the forming body. Molten glass delivered to the forming body trough via delivery vessel 40, exit conduit 44 and inlet conduit 50 overflows side walls of the trough and descends along the converging forming surfaces 54 as separate flows of molten glass. The separate flows of molten glass join below and along bottom edge 56 to produce a single ribbon of glass 58 that is drawn in a draw or flow direction 60 from bottom edge 56 by applying tension to the glass ribbon, such as by gravity, edge rolls 72 and pulling rolls 82, to control the dimensions of the glass ribbon as the glass cools and a viscosity of the glass increases. Accordingly, glass ribbon 58 goes through a visco-elastic transition and acquires mechanical properties that give the glass ribbon 58 stable dimensional characteristics. Glass ribbon 58 may, in some embodiments, be separated into individual glass sheets 62 by a glass separation apparatus 100 in an elastic region of the glass ribbon. A robot 64 may then transfer the individual glass sheets 62 to a conveyor system using gripping tool 65, whereupon the individual glass sheets may be further processed.

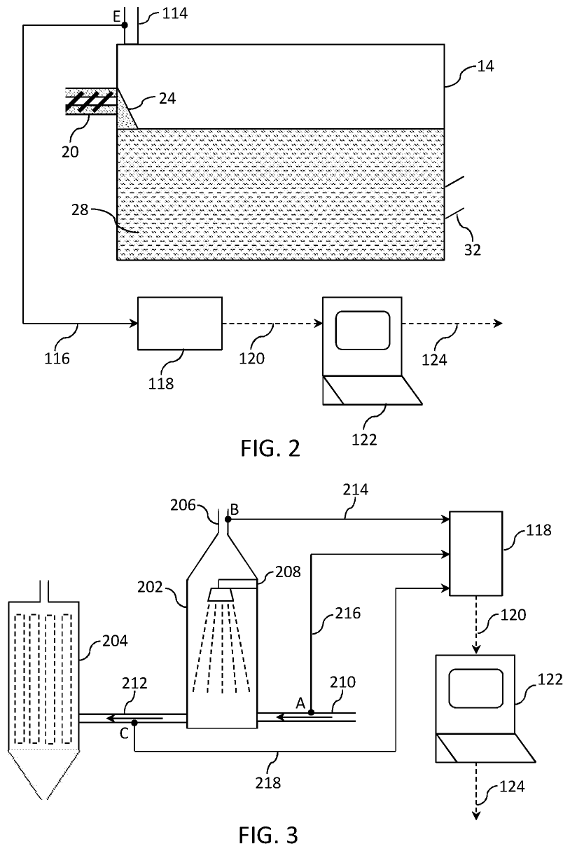

FIG. 2 shows an exemplary glass melting and gas sampling and analyzing system according to embodiments disclosed herein. As shown in FIG. 2, raw materials 24 are fed into melting vessel 14 from delivery device 20 and heated via heating elements (e.g., combustion burners or electrodes) so as to heat and convert the raw materials 24 into molten glass 28. In the process of heating and converting the raw materials 24 into molten glass 28, at least a portion of material initially present in raw materials 24 is converted to a gaseous phase during the melting operation. These and other gaseous species (e.g., gaseous species generated from combustion burners, etc.) present in the melting vessel 14 are vented from melting vessel 14 through exhaust port 114.

The gas composition exiting through exhaust port 114 can be sampled, for example, by a heated probe at sampling point E, by taking a plurality of measurements of the gas composition over a period of time. While not limited to a specific measurement frequency, the plurality of measurements may be taken at a frequency of at least one measurement per hour, such as at least 10 measurements per hour, and further such as at least 1 measurement per minute, and still further such as at least 10 measurements per minute, and yet still further such as at least 1 measurement per second, including from about 1 measurement per hour to about 10 measurements per second, including all ranges and sub-ranges there between.

The plurality of measurements may be taken using at least one measurement technique that detects and identifies gaseous species by at least one of type, amount, and concentration, as indicated in FIG. 2 by passage 116 of gas composition into gas composition measuring device 118. For example, in certain embodiments, the plurality of measurements are taken using Fourier transform infrared spectroscopy (FTIR). The plurality of measurements may also, for example, be taken by at least one other measurement technique, such as gas chromatography mass spectrometry (GCMS). The gas composition measuring device 118 can be configured to detect for at least one gaseous species present or suspected to be present in the gas composition, such as at least one gaseous species generated from a glass melt composition during a melting operation.

The plurality of measurements may then be sent to be analyzed by sending a measurement signal 120 to an analysis device 122, such as a computer, in order to determine an amount or concentration of at least one gaseous species, such as at least one gaseous species generated from a glass melt composition during a melting operation, in the gas composition as a function of time. The output data 124 from the analysis device 122 can then be subject to further review and analysis.

FIG. 3 shows an exemplary pollution abatement system that includes gas sampling and analysis according to embodiments disclosed herein. As shown in FIG. 3, a gas composition, such as an exhaust gas from a glass melting vessel, flows through conduit 210 and into spray tower 202. Such gas composition may, in certain exemplary embodiments, contain gas from a combination of sources, such as, for example, exhaust gas from a glass melting vessel in combination with air to dilute the exhaust gas.

Spray tower 202 includes a liquid injection port 208, through which a liquid, such as water or a solution containing a solvent, such as a lime-containing solvent, may be sprayed onto the gas composition in order to condense out pollutants that may be present in the gas composition. The cleaned air may exit through exhaust stack 206 whereas a discharge stream containing pollutants that were condensed in spray tower 202 may flow from spray tower 202 to bag house 204 through conduit 212. Bag house 204 can further filter out pollutants, such as particulates.

As illustrated in FIG. 3, gas compositions are sampled at three points, by, for example, heated probes located at sampling points A, B, and C. At each measurement point, a plurality of measurements may be taken using at least one measurement technique that detects and identifies gaseous species by at least one of type, amount, and concentration, as indicated in FIG. 3 by passage 214, 216, and 218 of gas composition into gas composition measuring device 118. For example, in certain embodiments, the plurality of measurements are taken using FTIR. The plurality of measurements may also, for example, be taken by at least one other measurement technique, such as GCMS. The gas composition measuring device 118 can be configured to detect for at least one gaseous species present or suspected to be present in the gas composition, such as at least one gaseous species generated from a glass melt composition during a melting operation.

The plurality of measurements may then be sent to be analyzed by sending a measurement signal 120 to an analysis device 122, such as a computer, in order to determine an amount or concentration of at least one gaseous species, such as at least one gaseous species generated from a glass melt composition during a melting operation, in the gas composition as a function of time. The output data 124 from the analysis device 122 can then be subject to further review and analysis.

While not limited to any particular species, the at least one gaseous species sampled and analyzed may, for example, be selected from at least one the group consisting of NO_{X}, SO_{X}, HCl, CO_{X}, HBr, and H₂O, wherein X is 1 or 2.

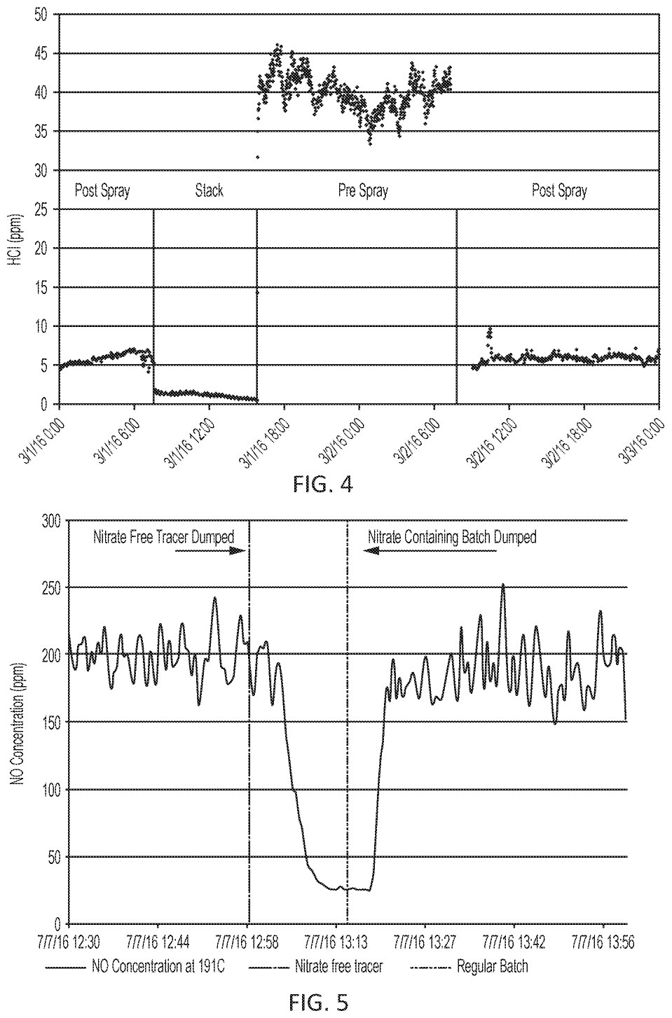

FIG. 4 shows a chart of HCl concentration as a function of time at three sampling points of an exemplary pollution abatement system, wherein the area identified as “Pre Spray” in FIG. 4 corresponds to sampling point A in FIG. 3, the area identified as “Stack” in FIG. 4 corresponds to sampling point B in FIG. 3, and the area identified as “Post Spray” in FIG. 4 corresponds to sampling point C in FIG. 3. As shown by FIG. 4, embodiments disclosed herein can be used to monitor the performance of a pollution abatement system as a function of time. Such monitoring could also be used to control the pollution abatement system by, for example, including appropriate computer controls for a feedback loop in accordance with methods known to those having ordinary skill in the art.

Embodiments disclosed herein can also be used to determine an amount or concentration of the at least one gaseous species in a gas composition that is not generated from a glass melt composition. For example, certain gaseous species present in a glass melting vessel may be generated from sources other than the glass melt composition, such as gaseous species generated from combustion burners as well as other gaseous species injected into or inherently present in the melting vessel.

Accordingly, embodiments disclosed herein include those in which a comparative melting operation is performed on a comparative composition that does not generate (or is not expected to generate) at least one gaseous species being analyzed. Such embodiments can include sampling a comparative gas composition generated from the comparative melting operation by taking a plurality of measurements of the comparative gas composition over a period of time. Such embodiments can further include analyzing the sampled comparative gas composition to determine an amount or concentration of the at least one gaseous species in the comparative gas composition as a function of time and comparing the amount or concentration of the at least one gaseous species in the comparative gas composition as a function of time with the amount or concentration of the at least one gaseous species in the gas composition as a function of time.

For example, embodiments disclosed herein can include feeding a comparative batch composition into a melting vessel and performing a melting operation on the comparative batch composition, wherein the comparative batch composition does not generate (or is not expected to generate) at least one gaseous species being analyzed. Before and/or after a melting operation is performed on the comparative batch composition, a melting operation is performed on a batch composition that is fed into the melting vessel and generates (or is expected to generate) at least one gaseous species. By sampling the gas composition that exits an exhaust port of the melting vessel during the melting operation performed on the comparative batch composition (that does not generate the at least one gaseous species) and sampling the gas composition that exits the exhaust port during the melting operation performed on the batch composition (that generates the at least one gaseous species), a comparison of the sampled compositions can help determine the amount or concentration of the at least one gaseous species exiting the exhaust port that is and is not generated from the glass melt composition.

FIG. 5 shows a chart of NO concentration as a function of time following introduction of nitrate-containing and nitrate-free batch into a glass melting vessel. In the embodiment shown in FIG. 5, about 10 pounds of nitrate-free batch were hand fed into a screw feeder at the time shown by the left most vertical line. The batch took about 6 minutes of travel time through the screw feeder barrel before entering the batch pile in the melting vessel, as shown by the timing of the abrupt drop in the NO concentration, which was measured using FTIR from a gas composition sampled from an exhaust port of the melting vessel using a heated probe. The measured NO concentration stayed at approximately 30 ppm for about 15 minutes, which was consistent with the feeding rate of about 40 pounds per hour. Then, nitrate-containing batch was fed to the screw feeder at the time shown by the right most vertical line. After the nitrate-containing batch reached the batch pile, the amount of NO abruptly increased to above 150 ppm. A comparison between the measured NO concentration with when the nitrate-free batch was in the melting vessel and when the nitrate-containing batch was in the melting vessel provides information regarding the concentration of NO in the gas composition that can be attributed to NO generated from the nitrate-containing batch relative to concentration of NO in the gas composition that can be attributed to NO generated from other sources, such as NO generated from combustion burners.

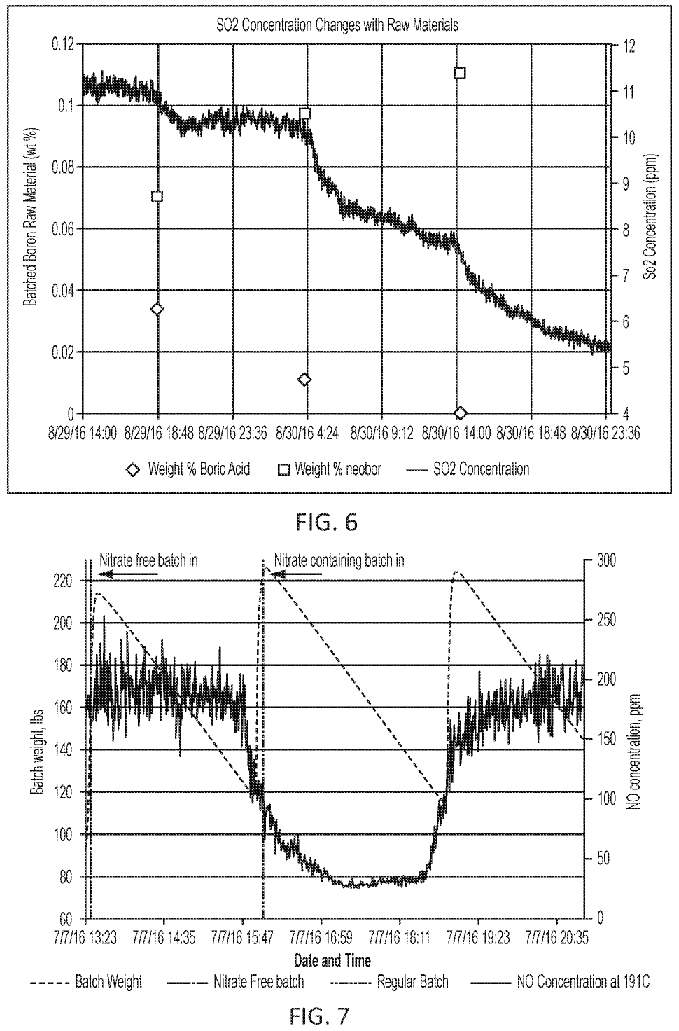

Embodiments disclosed herein can also provide information regarding the amount of gaseous species in a gas composition that can be attributed to different raw materials. For example, FIG. 6 shows a chart of measured SO₂ concentration as a function of time following introduction of boron-containing raw materials contaminated with different percentages of SO₂ into a glass melting vessel. Specifically, two different boron containing raw materials, Neobor and boric acid, were fed into a glass melting vessel at different weight ratios over different times and SO₂ concentration was measured using FTIR from a gas composition sampled from an exhaust port of the melting vessel using a heated probe. As can be seen from FIG. 6, a negative correlation exists between SO₂ and Neobor whereas a positive correlation exists between SO₂ and boric acid.

Accordingly, embodiments disclosed herein can further include correlating the amount or concentration of at least one gaseous species in a gas composition as a function of time to at least one attribute of a glass article made from the glass melt composition. For example, the presence of SO₂ in molten glass can be correlated to defects, such as blisters, in glass articles ultimately formed from the molten glass. Therefore, controlling the selection of source materials know to generate SO₂ based on the measured amount of SO₂ generated during a melting operation in accordance with embodiments disclosed herein can, in turn, affect quality attributes of ultimately produced glass articles. The same can also be done with respect to other gaseous species. Stated more broadly, embodiments disclosed herein include controlling a processing condition, such as selection of a raw material, of the glass melt composition in response to correlating the amount or concentration of the at least one gaseous species in the gas composition as a function of time to at least one attribute of a glass article made from the glass melt composition.

Embodiments disclosed herein can further include correlating the amount or concentration of at least one gaseous species in the gas composition as a function of time to at least one processing characteristic of the glass melt composition. For example, FIG. 7 shows a chart of NO concentration as a function of time following introduction of nitrate-containing and nitrate-free batch into a glass melting vessel. In the embodiment of FIG. 7, about 120 pounds of nitrate-free batch were placed at the top of storage bin (or batch feeder) at the time shown by the left-most vertical line. At a feed rate of about 40 pounds per hour, if the batch feeder was feeding in mass (plug) flow, the NO concentration in the gas composition should have begun dropping after about 3 hours and should have shown an abrupt drop, similar to FIG. 5. However, the NO concentration, which was measured using FTIR from a gas composition sampled from an exhaust port of the melting vessel using a heated probe, began gradually dropping after about 2.5 hours, showing that there was likely funnel flow through the middle of the batch feeder, which allowed nitrate-free batch to enter the melting vessel early. At the time indicated by the right-most vertical line, nitrate-containing batch was placed at the top of the batch feeder. As can be seen from FIG. 7, the NO concentration later rose gradually, again indicating funnel flow and batch mixing occurring in the batch feeder.

Accordingly, embodiments disclosed herein can provide diagnostic tools for understanding processing characteristics of glass melt compositions, such as batch feed characteristics, as shown and described with reference to FIG. 7. Other processing characteristics that may be correlated to concentration of at least one gaseous species in the gas composition as a function of time include, for example, volatile impurities in raw materials, oxidation state of the glass melt composition, water content in raw materials, changes in temperature of the batch pile and molten glass surface, changes in batch pile geometry and stability of batch pile shape, retention of volatile elements in the glass melt composition, leakage of air into the glass melting vessel, breakage of raw material delivery device (e.g., screw feeder), blockage of raw materials before entering glass melting vessel, and changes in melting vessel fill rate. In addition, changes in amounts or composition of at least one gaseous species can provide early warnings for appearance of solid or gaseous defects in the glass melt composition and provide clues to causes and timing of defect creation.

Moreover, embodiments disclosed herein include controlling a processing condition of the glass melt composition in response to correlating the amount or concentration of at least one gaseous species in the gas composition as a function of time to at least one processing characteristic of the glass melt composition. For example, in FIG. 7, the funnel flow processing characteristic may be at least partially addressed by at least one of controlling agitation of the batch feeder, adjusting a feed angle of the batch feeder, adjusting the particle size distribution of batch materials, using different batch materials, controlling the height of the batch in the batch feeder, controlling temperatures near the batch feeder, adjusting the design or geometry of the batch feeder, and adjusting the design of the raw material delivery device.

While the above embodiments have been described with reference to a fusion down draw process, it is to be understood that such embodiments are also applicable to other glass forming processes, such as float processes, slot draw processes, up-draw processes, and press-rolling processes.

It will be apparent to those skilled in the art that various modifications and variations can be made to embodiments of the present disclosure without departing from the spirit and scope of the disclosure. Thus it is intended that the present disclosure cover such modifications and variations provided they come within the scope of the appended claims and their equivalents.

1. A method for analyzing a glass melt composition comprising:
sampling a gas composition comprising at least one gaseous species generated from the glass melt composition during a melting operation by taking a plurality of measurements of the gas composition over a period of time; and
analyzing the sampled composition to determine an amount or concentration of the at least one gaseous species in the gas composition as a function of time.

2. The method of claim 1, wherein the plurality of measurements are taken from an exhaust port of a glass melting vessel.

3. The method of claim 1, wherein the plurality of measurements are taken from at least one point in a pollution abatement system.

4. The method of claim 1, wherein the plurality of measurements are taken using Fourier transform infrared spectroscopy (FTIR).

5. The method of claim 1, wherein the plurality of measurements are taken using gas chromatography mass spectrometry (GCMS).

6. The method of claim 1, wherein the at least one gaseous species is selected from the group consisting of NO_{X}, SO_{X}, HCl, CO_{X}, HBr, and H₂O, wherein X is 1 or 2.

7. The method of claim 1, wherein the method further comprises determining the amount or concentration of the at least one gaseous species in the gas composition that is not generated from the glass melt composition.

8. The method of claim 7, wherein the method comprises performing a comparative melting operation on a comparative composition that does not generate the at least one gaseous species, sampling a comparative gas composition generated from the comparative melting operation by taking a plurality of measurements of the comparative gas composition over a period of time, analyzing the sampled comparative gas composition to determine an amount or concentration of the at least one gaseous species in the comparative gas composition as a function of time, and comparing the amount or concentration of the at least one gaseous species in the comparative gas composition as a function of time with the amount or concentration of the at least one gaseous species in the gas composition as a function of time.

9. The method of claim 1, wherein the method further comprises correlating the amount or concentration of the at least one gaseous species in the gas composition as a function of time to at least one attribute of a glass article made from the glass melt composition.

10. The method of claim 9, wherein the method further comprises controlling a processing condition of the glass melt composition in response to correlating the amount or concentration of the at least one gaseous species in the gas composition as a function of time to at least one attribute of the glass article made from the glass melt composition.

11. The method of claim 1, wherein the method further comprises correlating the amount or concentration of the at least one gaseous species in the gas composition as a function of time to at least one processing characteristic of the glass melt composition.

12. The method of claim 11, wherein the method further comprises controlling a processing condition of the glass melt composition in response to correlating the amount or concentration of the at least one gaseous species in the gas composition as a function of time to at least one processing characteristic of the glass melt composition.

13. A method of making a glass article comprising:
sampling a gas composition comprising at least one gaseous species generated from a glass melt composition during a melting operation by taking a plurality of measurements of the gas composition over a period of time;
analyzing the sampled composition to determine an amount or concentration of the at least one gaseous species in the gas composition as a function of time; and
forming the glass article from the glass melt composition.

14. The method of claim 13, wherein the plurality of measurements are taken from an exhaust port of a glass melting vessel.

15. The method of claim 13, wherein the plurality of measurements are taken from at least one point in a pollution abatement system.

16. The method of claim 13, wherein the plurality of measurements are taken using Fourier transform infrared spectroscopy (FTIR).

17. The method of claim 13, wherein the plurality of measurements are taken using gas chromatography mass spectrometry (GCMS).

18. The method of claim 13, wherein the at least one gaseous species is selected from the group consisting of NO_{X}, SO_{X}, HCl, CO_{X}, HBr, and H₂O, wherein X is 1 or 2.

19. The method of claim 13, wherein the method further comprises correlating the amount or concentration of the at least one gaseous species in the gas composition as a function of time to at least one attribute of the glass article.

20. The method of claim 19, wherein the method further comprises controlling a processing condition of the glass melt composition in response to correlating the amount or concentration of the at least one gaseous species in the gas composition as a function of time to at least one attribute of the glass article.

21. The method of claim 13, wherein the method further comprises correlating the amount or concentration of the at least one gaseous species in the gas composition as a function of time to at least one processing characteristic of the glass melt composition.

22. The method of claim 21, wherein the method further comprises controlling a processing condition of the glass melt composition in response to correlating the amount or concentration of the at least one gaseous species in the gas composition as a function of time to at least one processing characteristic of the glass melt composition.

23. A glass article made by the method of claim 13.

24. An electronic device comprising the glass article of claim 23.
